(19) United States
(12) Patent Application Publication
(10) Pub. No.: US 2025/0180683 A1
(43) Pub. Date: Jun. 5, 2025

(54) MAGNETIC RESONANCE IMAGING APPARATUS, CHEMICAL SHIFT PEAK DETECTION METHOD, AND CONTRAST AGENT NANOPARTICLE

(71) Applicant: CANON MEDICAL SYSTEMS CORPORATION, Otawara-shi (JP)

(72) Inventor: Sotaro TAKI, Chofu Tokyo (JP)

(73) Assignee: CANON MEDICAL SYSTEMS CORPORATION, Otawara-shi (JP)

(21) Appl. No.: 18/947,639

(22) Filed: Nov. 14, 2024

(30) Foreign Application Priority Data
Nov. 30, 2023 (JP) 2023-202551

(51) Int. Cl. G01R 33/56; G01N 33/543; G01R 33/485

(52) U.S. Cl. CPC G01R 33/5601; G01N 33/5432; G01N 33/54346; G01R 33/485

(57) ABSTRACT

A magnetic resonance imaging apparatus according to the embodiment includes a stimulation-imparting unit; sequence control circuitry; and processing circuitry, in which the stimulation-imparting unit is configured to apply a dissolving stimulus to contrast agent nanoparticles accumulated in an imaging region after the contrast agent nanoparticles are injected into a subject, the sequence control circuitry is configured to collect a first group of magnetic resonance signals by CEST imaging while changing conditions of a saturation pulse before being contrasted with the contrast agent nanoparticles; and collect a second group of magnetic resonance signals by the CEST imaging while the conditions of the saturation pulse are changed after being contrasted with the contrast agent nanoparticles and after the dissolving stimulus by the stimulation-imparting unit is applied, the processing circuitry is configured to calculate a difference between a first Z spectrum generated on the basis of the first group of magnetic resonance signals and a second Z spectrum generated on the basis of the second group of magnetic resonance signals; and detect a plurality of peaks indicating a decrease in a magnetic resonance signal due to a chemical shift on the basis of the difference.

### CROSS-REFERENCE TO RELATED APPLICATION

The present application claims priority is claimed on Japanese Patent Application No. 2023-202551, filed Nov. 30, 2023, the content of which is incorporated herein by reference.

### FIELD

### Field of the Invention

The embodiments disclosed in this specification and the drawings relate to a magnetic resonance imaging apparatus, a chemical shift peak detection method, and a contrast agent nanoparticle.

### Background

Protons solutes present in solutes in water have a specific resonant frequency for each proton solute, and are chemically exchanged with protons in the water (chemical exchange). Therefore, when a saturation pulse is selectively sent to the protons of the solute, saturated protons are exchanged for unsaturated protons in the water through a chemical exchange phenomenon, and the saturated protons are transferred to the water (saturation transfer). This phenomenon is collectively called chemical exchange saturation transfer (CEST), and since a rate Kₑₓ of chemical exchange in the CEST depends on temperature and pH, it can be used to measure them.

Imaging methods utilizing the CEST phenomenon (for example, MR imaging methods utilizing exchange of protons in amide groups (—C(═O)—NH—), hydroxyl groups (—OH), and amino groups (—NH₂) with protons in free water) is called CEST imaging (CEST imaging). At this time, a material that realizes the CEST phenomenon (hereinafter, referred to as a CEST substance) is used in the CEST imaging.

FIG. 3 is a diagram describing an overview of the CEST. FIG. 3A to B show a process of selectively saturating the protons of a CEST substance, and FIG. 3 B to C show a process of exchanging the saturated protons of the CEST substance with protons of free water.

The CEST phenomenon reflects properties of a substance such as temperature, pH, or the like, that is, the rate of chemical exchange in the CEST phenomenon depends on temperature and pH. Therefore, CEST imaging can be used to measure temperature and pH. For example, by combining a CEST substance having two proton pools with a technique known as a ratiometric method, it is possible to perform pH imaging that captures pH regardless of a concentration of the CEST substance. In calculating pH using the ratiometric method, for example, in a Z spectrum showing the influence of a CEST effect, signal values at two peaks relating to two proton pools and signal values used to normalize the signal values in the Z spectrum are used. To obtain data used to generate the Z spectrum, a magnetic resonance imaging technique for the CEST effect (hereinafter, referred to as CEST imaging) is performed.

FIG. 4 shows an example of an overview of conventional CEST imaging, generation of a Z spectrum, and detection of peaks in a CEST substance. The CEST substance shown in FIG. 4 is iopamidol, which corresponds to two kinds of substances, two amide groups corresponding to a chemical shift of 4.2 ppm and one amide group corresponding to a chemical shift of 5.6 ppm. In FIG. 4, frequencies of a plurality of saturation pulses used in the CEST imaging are in a range from −10 ppm to 10 ppm in increments of 0.1 ppm. For example, when a static magnetic field strength is 3 T, the frequency of the saturation pulse corresponding to 0 ppm is a resonance frequency (hereinafter, referred to as a center frequency) of free water based on the static magnetic field strength, which is 128 MHz. In this case, the frequency of the saturation pulse corresponding to +10 ppm is 128 MHz+128×10 Hz. On the other hand, the frequency of the saturation pulse corresponding to −10 ppm is 128 MHz−128×10 Hz.

As shown in FIG. 4, according to the CEST imaging, an MR image (a CEST image) is acquired by applying a plurality of saturation pulses having a center frequency of 0 ppm and varying in increments of 0.1 ppm from −10 ppm to +10 ppm. Signal data is acquired on the basis of pixel values within regions of interest (ROIs) (ROI1 and ROI2 in FIG. 4) in 201 MR images. A Z spectrum is generated on the basis of the acquired signal data. Then, a signal value (a peak) at a chemical shift (ppm) relating to the CEST substance in the Z spectrum is detected.

As shown in FIG. 4, in the conventional CEST imaging, there is a problem that an imaging time is long in order to obtain as many as 201 CEST images. In addition, when a Z spectrum with less noise is obtained, there is a problem in that the imaging time is further increased due to the generation of a plurality of Z spectra. In addition, as shown in FIG. 4, in the detection of the two peaks of the CEST substance in the Z spectrum, when all natural proton pools in a living body are taken into consideration, fitting using five approximate equations (two approximate equations corresponding to the two peaks of the two CEST substances, an approximate equation of a peak for water, an approximate equation for a magnetization transfer (MT) effect, and an approximate equation for an Overhauser effect) is required. The fitting using the five approximation equations requires complicated calculations, takes time, and may result in poor fitting accuracy. In addition, since initial values of parameters in the five approximate equations differ according to the tissue from which the Z spectrum is obtained, the initial values must be set according to the tissue.

An enhanced permeability and retention (EPR) effect is a phenomenon in which the permeability of blood vessels to substances and the retention of permeated substances are enhanced in tumor tissue. As shown in FIG. 5, blood vessels 1101 near a tumor 1103 have large gaps (150 nm or more), and substances flowing in the blood leak out and accumulate in the tumor tissue (EPR effect), whereas blood vessels 1102 in normal tissue 1104 have small gaps (approximately 5 to 50 nm), and necessary nutrients and the like are delivered to the normal tissue 1104 through the blood flow.

Furthermore, in order to achieve the EPR effect, it has been proposed to incorporate various substances into nanoparticles, and nanoparticles 1201 incorporating a CEST substance 1202 (for example, iopamidol) as shown in FIG. 6 have also been proposed.

An imaging apparatus and an imaging method using a CEST substance as a contrast agent require a certain amount of the substance that induces the CEST phenomenon. In the case of a method in which a CEST substance is incorporated into nanoparticles, the nanoparticles may break down in the blood, or the nanoparticles may not break down in a target tumor, the release of the CEST substance is delayed, therefore, it takes time for the CEST substance to accumulate in the tumor, and imaging takes a long time when performed after waiting for the accumulation.

### BRIEF DESCRIPTION OF THE DRAWINGS

FIG. 1 is a diagram showing an overview of a chemical shift peak detection method according to an embodiment.

FIG. 2 is a flowchart showing an example of a procedure of the chemical shift peak detection method according to the embodiment.

FIG. 3 is a diagram showing a CEST phenomenon according to the related art.

FIG. 4 is a diagram showing an example of an overview of conventional CEST imaging, generation of a Z spectrum, and detection of peaks in a CEST substance according to the related art.

FIG. 5 is a diagram showing an EPR effect according to the related art.

FIG. 6 is a diagram showing an example of nanoparticles incorporating a CEST substance according to the related art.

FIG. 7 is a block diagram showing a configuration of an MRI apparatus according to an embodiment.

FIG. 8 is a flowchart showing an example of a procedure of a state quantity determination process according to the embodiment.

FIG. 9 is a diagram showing an example of a non-contrast Z spectrum according to the embodiment.

FIG. 10 is a diagram showing an example of a contrast Z spectrum according to the embodiment.

FIG. 11 is a diagram showing an example of a difference spectrum according to the embodiment.

FIG. 12 is a diagram showing an example in which function fitting is performed on the difference spectrum according to the embodiment.

FIG. 13 is a diagram showing an example of the difference spectrum after integration according to the embodiment.

FIG. 14 is a diagram showing an example of an effect of the embodiment.

### DETAILED DESCRIPTION

Hereinafter, with reference to the drawings, embodiments of a magnetic resonance imaging apparatus (hereinafter, referred to as an MRI apparatus), a chemical shift peak detection method, and a contrast agent nanoparticle will be described. The embodiments are not limited to the following embodiments. Furthermore, the contents described in each of the embodiments can, in principle, be similarly applied to other embodiments. In the following embodiments, parts with the same reference symbols perform similar operations, and duplicated description thereof will be omitted as appropriate.

A magnetic resonance imaging apparatus according to this embodiment includes a stimulation-imparting unit; sequence control circuitry; and processing circuitry. The stimulation-imparting unit is configured to apply a dissolving stimulus to contrast agent nanoparticles accumulated in an imaging region after the contrast agent nanoparticles are injected into a subject. The sequence control circuitry is configured to collect a first group of magnetic resonance signals by CEST imaging while changing conditions of a saturation pulse before being contrasted with the contrast agent nanoparticles; and collect a second group of magnetic resonance signals by the CEST imaging while changing the conditions of the saturation pulse after being contrasted with the contrast agent nanoparticles and after the dissolving stimulus by the stimulation-imparting unit is applied. The processing circuitry is configured to calculate a difference between a first Z spectrum generated on the basis of the first group of magnetic resonance signals and a second Z spectrum generated on the basis of the second group of magnetic resonance signals; and detect a plurality of peaks indicating a decrease in a magnetic resonance signal due to a chemical shift on the basis of the difference.

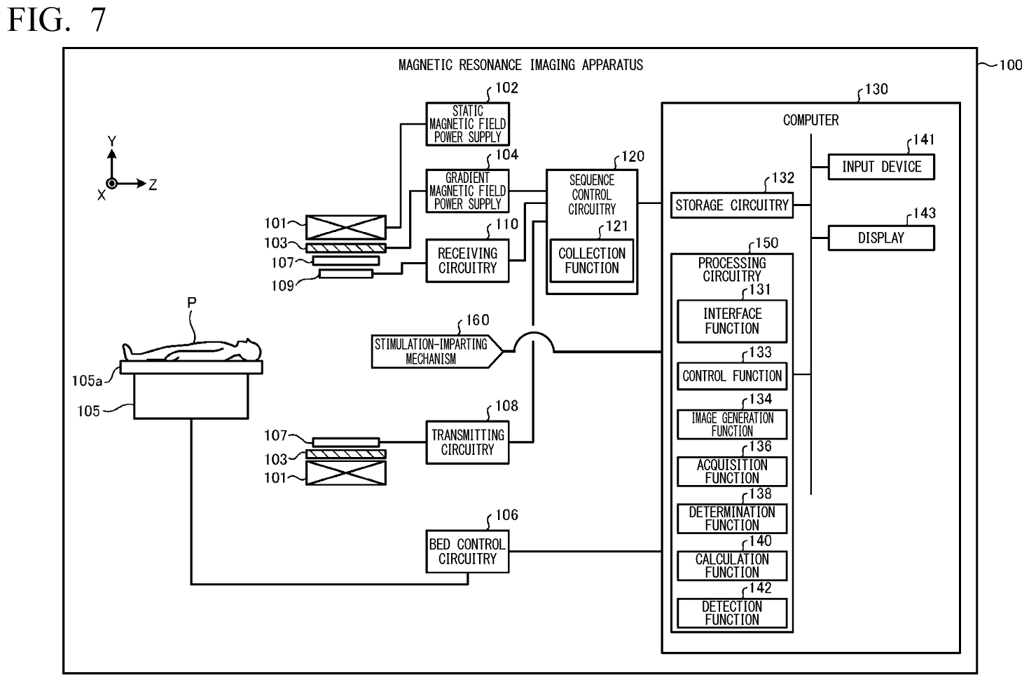

FIG. 7 is a block diagram showing the configuration of an MRI apparatus 100 according to an embodiment. As shown in FIG. 7, the MRI apparatus 100 includes a static magnetic field magnet 101, a static magnetic field power supply 102, a gradient magnetic field coil 103, a gradient magnetic field power supply 104, a bed 105, bed control circuitry 106, a transmitting coil 107, transmitting circuitry 108, a receiving coil 109, receiving circuitry 110, sequence control circuitry 120, a computer 130 (also referred to as an image-processing device), and a stimulation-imparting mechanism 160. The MRI apparatus 100 does not include a subject P (for example, a human body). Further, the configuration shown in FIG. 7 is merely one example. For example, the sequence control circuitry 120 and various parts in the computer 130 may be integrated or separated as appropriate. The computer 130 is mounted in, for example, a console. For example, the stimulation-imparting mechanism 160 may be disposed away from the subject P as shown in the drawing, or may be disposed in close contact with the subject P.

The static magnetic field magnet 101 is a magnet formed in a hollow and approximately cylindrical in shape, and generates a static magnetic field in an internal space thereof. The static magnetic field magnet 101 is, for example, a superconducting magnet or the like, and is excited by receiving a current from the static magnetic field power supply 102. The static magnetic field power supply 102 supplies a current to the static magnetic field magnet 101. The static magnetic field magnet 101 may be a permanent magnet, and in this case, the MRI apparatus 100 does not need to include the static magnetic field power supply 102. In addition, the static magnetic field power supply 102 may be provided separately from the MRI apparatus 100.

The gradient magnetic field coil 103 is a coil formed in a hollow and approximately cylindrical in shape, and is disposed inside the static magnetic field magnet 101. The gradient magnetic field coil 103 is formed by combining three coils corresponding to mutually orthogonal X, Y, and Z axes, and these three coils are individually supplied with a current from the gradient magnetic field power supply 104 to generate a gradient magnetic field of which a magnetic field strength changes along each of the X, Y, and Z axes. The gradient magnetic fields of the X, Y, and Z axes generated by the gradient magnetic field coil 103 are, for example, a slice gradient magnetic field Gs, a phase encoding gradient magnetic field Ge, and a readout gradient magnetic field Gr. The gradient magnetic field power supply 104 supplies a current to the gradient magnetic field coil 103.

The bed 105 includes a top plate 105a on which the subject P is placed, and inserts the top plate 105a into a cavity (an imaging port) of the gradient magnetic field coil 103 with the subject P placed thereon under the control of the bed control circuitry 106. Typically, the bed 105 is mounted so that a longitudinal direction thereof is parallel to a central axis of the static magnetic field magnet 101. Under the control of the computer 130, the bed control circuitry 106 drives the bed 105 to move the top plate 105a in the longitudinal direction and a vertical direction.

The transmitting coil 107 is disposed inside the gradient magnetic field coil 103, and receives RF pulses from the transmitting circuitry 108 to generate a high-frequency magnetic field. The transmitting circuitry 108 supplies, to the transmitting coil 107, an RF pulse corresponding to a Larmor frequency determined by a type of target atom and a magnetic field strength.

The receiving coil 109 is disposed inside the gradient magnetic field coil 103 and receives a magnetic resonance signal (hereinafter, referred to as an MR signal) emitted from the subject P due to an influence of a high-frequency magnetic field. When the receiving coil 109 receives the MR signal, it outputs the received MR signal to the receiving circuitry 110.

The above-described transmitting coil 107 and receiving coil 109 are merely examples. The transmitting coil 107 and the receiving coil 109 may be configured by combining one or more of a coil having only a transmitting function, a coil having only a receiving function, or a coil having a transmitting and receiving function.

The receiving circuitry 110 detects the MR signal output from the receiving coil 109, and generates MR data on the basis of the detected MR signal. Specifically, the receiving circuitry 110 generates MR data by digitally converting the MR signal output from the receiving coil 109. The receiving circuitry 110 also transmits the generated MR data to the sequence control circuitry 120. The receiving circuitry 110 may be provided on the side of a gantry device that includes the static magnetic field magnet 101, the gradient magnetic field coil 103, and the like.

The sequence control circuitry 120 has a collection function 121, and the collection function 121 performs imaging of the subject P by driving the gradient magnetic field power supply 104, the transmitting circuitry 108, and the receiving circuitry 110 on the basis of sequence information transmitted from the computer 130. Here, the sequence information is information that defines a procedure for performing imaging, and is also called a sequence condition. The sequence information defines the strength of a current supplied to the gradient magnetic field coil 103 by the gradient magnetic field power supply 104 and the timing of supplying the current, the strength of the RF pulse supplied to the transmitting coil 107 by the transmitting circuitry 108 and a timing of applying the RF pulse, a timing at which the receiving circuitry 110 detects the MR signal, and the like.

The sequence control circuitry 120 is, for example, integrated circuitry such as an application-specific integrated circuit (ASIC) and a field-programmable gate array (FPGA), or an electronic circuit such as a central processing unit (CPU) and a micro processing unit (MPU). The sequence control circuitry 120 corresponds to a sequence control unit. Moreover, the sequence control circuitry 120 that realizes the collection function 121 corresponds to a collection unit.

The collection function 121 collects a first group of magnetic resonance signals, while changing saturation pulse conditions for a frequency band determined by a determination function 138 described below, by CEST imaging before the subject P is contrasted with a contrast agent (a contrast substance) (hereinafter, referred to as a non-contrast CEST imaging). In addition, the collection function 121 collects a second group of magnetic resonance signals, while changing the saturation pulse conditions for the frequency band determined by the determination function 138, by the CEST imaging after the subject P is contrasted with the contrast agent (hereinafter, referred to as contrast CEST imaging). As for the sequences relating to the non-contrast CEST imaging and the contrast CEST imaging, known sequences can be used except that the frequency of the saturation pulse is within the frequency band determined above, and thus a description thereof will be omitted. Also, the CEST imaging and the frequency band will be described below. The collection function 121 drives the gradient magnetic field power supply 104, the transmitting circuitry 108, and the receiving circuitry 110 to image the subject P, thus receives MR data from the receiving circuitry 110, and then transfers the received MR data to the computer 130.

The computer 130 performs overall control of the MRI apparatus 100, generates images, and the like. The computer 130 includes a storage circuitry 132, an input device 141, a display 143, and processing circuitry 150. The processing circuitry 150 has an interface function 131, a control function 133, an image generation function 134, an acquisition function 136, a determination function 138, a calculation function 140, and a detection function 142.

Each of the processing functions performed by the interface function 131, the control function 133, the image generation function 134, the acquisition function 136, the determination function 138, the calculation function 140, and the detection function 142 is stored in the storage circuitry 132 in the form of a program executable by the computer 130. The processing circuitry 150 is a processor that realizes a function corresponding to each of the programs by reading the program from the storage circuitry 132 and executing the program. In other words, the processing circuitry 150 in a state in which each of the programs has been read has each of the functions shown in the processing circuitry 150 in FIG. 7.

In FIG. 7, although it has been described that the processing functions performed by the interface function 131, the control function 133, the image generation function 134, the acquisition function 136, the determination function 138, the calculation function 140, and the detection function 142 are realized by single processing circuitry 150, the processing circuitry 150 may be configured by combining a plurality of independent processors, and each of the processors may execute a program to realize the functions. In other words, each of the above-described functions may be configured as a program and one processing circuitry 150 may execute each of the programs, or a specific function may be implemented in a dedicated and independent program execution circuitry.

The term “processor” used in the above description refers to circuitry such as a CPU, a graphical processing unit (GPU), application-specific integrated circuitry, and a programmable logic device (for example, a simple programmable logic device (SPLD), a complex programmable logic device (CPLD), and a field programmable gate array (FPGA)). The processor realizes functions by reading and executing programs stored in the storage circuitry 132.

The program may be directly embedded in the circuitry of the processor instead of storing the program in the storage circuitry 132. In this case, the processor realizes the function by reading and executing the program embedded in the circuitry. Similarly, the bed control circuitry 106, the transmitting circuitry 108, the receiving circuitry 110, the sequence control circuitry 120, and the like are also configured by electronic circuitry such as the above-described processor.

The storage circuitry 132 stores the MR data received by the processing circuitry 150 having the interface function 131, various data acquired by the acquisition function 136, various image data generated by the image generation function 134, a calculation process used in the calculation function 140, a difference calculated by the calculation process, a detection process used in the detection function 142, a plurality of peaks determined by the detection function 142, and the like.

The storage circuitry 132 also stores MR data (also referred to as k-space data) arranged in a k-space by the control function 133. The various types of data stored will be described below. For example, the storage circuitry 132 is realized by a semiconductor memory element such as a random access memory (RAM) or a flash memory, a hard disk, an optical disk, or the like. The storage circuitry 132 may be referred to as a memory.

The input device 141 accepts various instructions and information input from a user. The input device 141 is realized, for example, by a trackball, a switch button, a mouse, a keyboard, a touchpad that performs an input operation by touching an operating surface, a touch screen in which a display screen and a touchpad are integrated, non-contact input circuitry using an optical sensor, and a voice input circuitry. The input device 141 is electrically connected to the processing circuitry 150, converts an input operation received from the user into an electrical signal, and outputs the electrical signal to the processing circuitry 150.

In this specification, the input device 141 is not limited to a device equipped with a physical operation part (an input interface) such as a mouse and a keyboard. For example, an example of the input device 141 also includes electrical signal processing circuitry that receives an electrical signal corresponding to an input operation from an external input device provided separately from the MRI apparatus 100 and outputs the electrical signal to control circuitry. The input device 141 corresponds to an input unit and may be referred to as an input interface.

The display 143 displays a graphical user interface (GUI) for accepting an input of imaging conditions, and the like, or an image generated by the processing circuitry 150 having the image generation function 134 under the control of the processing circuitry 150 having the control function 133. The display 143 is realized by, for example, a display device such as a CRT display, a liquid crystal display, an organic EL display, an LED display, a plasma display, or any other display or monitor known in the art.

An example of the CEST imaging according to this embodiment will be described below. In the CEST imaging, the sequence control circuitry 120 applies a saturation pulse, which is a frequency-selective radio frequency (RF) pulse, at a frequency (an off-resonance frequency) away from a resonance frequency of free water and at a resonance frequency of exchangeable protons (for example, protons of a compound), to the subject P before the MR signal is collected. The saturation pulse is also called a presaturation pulse. The CEST imaging is a magnetic resonance imaging technique that applies a plurality of saturation pulses and collects a plurality of magnetic resonance signals (MR signals) according to the plurality of saturation pulses. A frequency of each of the plurality of saturation pulses used in the CEST imaging is determined by the determination function 138, described below. In the frequencies of the plurality of saturation pulses, a frequency interval between two adjacent saturation pulses is explained as 0.1 ppm. The frequency interval between the two adjacent saturation pulses is not limited to 0.1 ppm, and can be set arbitrarily as long as a Z spectrum that clearly indicates the CEST phenomenon can be depicted.

For the sake of concrete explanation, it is assumed below that a plurality of substances (a plurality of CEST substances: proton pools) that realize the CEST phenomenon are included in the contrast agent. In other words, the contrast agent includes a plurality of substances (functional groups) that have different chemical shifts. For example, when the contrast agent is iopamidol, iopamidol contains two substances (two types of amide groups) of which chemical shifts are different from each other. Specifically, the two substances contained in iopamidol correspond to two types of substances including two amide groups corresponding to a chemical shift of 4.2 ppm, and one amide group corresponding to a chemical shift of 5.6 ppm. The contrast agent in this embodiment is not limited to iopamidol, and any contrast agent can be used as long as it has a plurality of CEST substances having different chemical shifts.

The contrast agent according to the embodiment is used in the form of contrast agent nanoparticles in which the contrast agent is incorporated in stimulus-sensitive nanoparticles.

The stimulus-sensitive nanoparticles are preferably liposomes, and the liposomes are preferably surface-modified. As the surface modification, for example, polyethylene glycol (PEG) modification is preferable since it can improve stability of the liposome in blood, and modification with a metal, a metal complex, or a metal ion is preferable since it can more easily monitor accumulation of the contrast agent nanoparticles at an imaging target site, and modification with a metal ion that significantly changes a relaxation time of the contrast agent nanoparticles is more preferable.

A diameter of the contrast agent nanoparticles is not particularly limited, but is preferably 10 to 100 nm, and more preferably 50 to 100 nm. When the diameter of the contrast agent nanoparticles is 10 nm or more, in the blood vessels of normal tissue, it is difficult for the contrast agent nanoparticles to pass through gaps and thus they are unlikely to escape from the blood vessels, whereas in the blood vessels near a tumor, it is easy for the contrast agent nanoparticles to pass through gaps and thus they are likely to escape from the blood vessels and to accumulate in the tumor (EPR effect). The diameter of the contrast agent nanoparticles is an arithmetic mean particle diameter calculated by measuring particle diameter distribution using a laser method.

The processing circuitry 150 transmits sequence information to the sequence control circuitry 120 and receives MR data from the sequence control circuitry 120 through the interface function 131. Furthermore, when the processing circuitry 150 having the interface function 131 receives the MR data, it stores the received MR data in the storage circuitry 132. The processing circuitry 150 that realizes the interface function 131 corresponds to an interface unit.

The processing circuitry 150 performs overall control of the MRI apparatus 100 using the control function 133, and controls imaging, image generation, image display, and the like. For example, the processing circuitry 150 having the control function 133 receives an input of imaging conditions (imaging parameters, or the like) on a GUI, and generates sequence information according to the saturation pulse conditions set by the received imaging conditions. In addition, the processing circuitry 150 having the control function 133 transmits the generated sequence information to the sequence control circuitry 120.

For example, the control function 133 transmits sequence information for CEST imaging including a plurality of frequencies related to saturation pulses in the frequency band determined by the determination function 13, and sequence information for MR imaging (hereinafter, referred to as map imaging) that collects MR data related to the generation of a B₀ map to the sequence control circuitry 120. The processing circuitry 150 that realizes the control function 133 corresponds to a control unit.

The processing circuitry 150 reads k-space data from the storage circuitry 132 using the image generation function 134, and performs a reconstruction process such as Fourier transform on the read k-space data to generate an image. For example, the image generation function 134 generates a B₀ map on the basis of the MR data (hereinafter, referred to as map MR data) collected by map imaging. The B₀ map is a map that indicates inhomogeneity of a static magnetic field (B₀) in an imaging region. The image generation function 134 stores the generated B₀ map in the storage circuitry 132. Since the generation of the B₀ map can appropriately using a known method, a description thereof will be omitted. The processing circuitry 150 that realizes the image generation function 134 corresponds to an image generation unit.

The processing circuitry 150 generates, through the image generation function 134, a first Z spectrum on the basis of the first group of magnetic resonance signals. Specifically, the image generation function 134 generates a plurality of MR images (hereinafter, referred to as non-contrast MR images) on the basis of the first group of magnetic resonance signals (non-contrast data) collected by pre-contrast (non-contrast) CEST imaging. The image generation function 134 generates the first Z spectrum (the non-contrast Z spectrum) on the basis of a plurality of non-contrast MR images and a B₀ map, with correction (hereinafter, referred to as B₀ correction) of a position of the saturation pulse. The B₀ correction may be performed on the contrast MR images. At this time, the image generation function 134 generates the first Z spectrum on the basis of a plurality of contrast MR images on which the B₀ correction has been performed. At this time, the first Z spectrum is normalized by a reference MR signal which will be described below. That is, the first Z spectrum is generated on the basis of the first group of magnetic resonance signals and the reference MR signal. A known method can be applied to generate the Z spectrum, and thus a description thereof will be omitted. The B₀ correction may be omitted.

The processing circuitry 150 generates, through the image generation function 134, a second Z spectrum on the basis of the second group of magnetic resonance signals. Specifically, the image generation function 134 generates a plurality of MR images (hereinafter, referred to as contrast MR images) on the basis of the second group of magnetic resonance signals (contrast data) collected by post-contrast CEST imaging. The image generation function 134 generates the second Z spectrum (contrast Z spectrum) on the basis of the plurality of contrast MR images and the B₀ map, with B₀ correction. The B₀ correction may be performed on the contrast MR images. At this time, the image generation function 134 generates the second Z spectrum on the basis of the plurality of contrast MR images on which the B₀ correction has been performed. At this time, the second Z spectrum is normalized by a reference MR signal which will be described below. That is, the second Z spectrum is generated on the basis of the second group of magnetic resonance signals and the reference MR signal.

The first Z spectrum and the second Z spectrum are Z spectra in which a position of the saturation pulse in the CEST imaging is corrected on the basis of a B₀ map generated by imaging other than the CEST imaging, and normalized by a reference MR signal. The processing circuitry 150 that realizes the image generation function 134 corresponds to an image generation unit. The first Z spectrum and the second Z spectrum may be generated by the calculation function 140.

The processing circuitry 150 acquires information about the contrast agent including a plurality of substances having different chemical shifts by the acquisition function 136. Specifically, the acquisition function 136 acquires information about the contrast agent input by the user via the input device 141. The information about the contrast agent is, for example, a plurality of chemical shifts (ppm) corresponding to a plurality of CEST substances (substances that realize the CEST phenomenon). When a name of the contrast agent is input by the user via the input device 141, the acquisition function 136 acquires information about the contrast agent by comparing the input name of the contrast agent with a correspondence table stored in the storage circuitry 132. The correspondence table corresponds to, for example, a lookup table in which the name of the contrast agent corresponds to the plurality of chemical shifts (ppm). The processing circuitry 150 that realizes the acquisition function 136 corresponds to an acquisition unit.

The processing circuitry 150 determines, through the determination function 138, the frequency band related to a decrease in the MR signal due to the chemical shift on the basis of the information acquired by the acquisition function 146. The frequency band is centered on each of the plurality of chemical shifts and corresponds to a region of the frequency of the saturation pulse related to the decrease in the MR signal due to the CEST phenomenon for each of the plurality of CEST substances. The frequency band (which may also be referred to as a frequency domain) is a predetermined range determined on the order of ppm, with a saturation frequency of water being 0 ppm, and has a predetermined width centered on a specific value (the chemical shift) determined by components of the contrast agent. For example, when the plurality of chemical shifts are 4.2 ppm and 5.6 ppm, the determination function 138 determines a band including a predetermined frequency range centered on 4.2 ppm and 5.6 ppm as the frequency band. The predetermined frequency range is, for example, +1 ppm. The predetermined range is not limited to +1 ppm, and can be set arbitrarily as long as it is within the frequency range related to the decrease in the MR signal due to the CEST phenomenon of each of the plurality of CEST substances.

The determination function 138 may use the number n of times to apply a saturation pulse in a +ppm or −ppm direction centered on each of the plurality of chemical shifts, instead of the predetermined frequency range. For example, when n=10, the frequency interval between two adjacent saturation pulses is 0.1 ppm, and thus the frequency range is the same as above.

Also, the determination function 138 may determine the frequency band by further using a B₀ map generated before the execution of the CEST imaging. Specifically, the determination function 138 determines an average value of a plurality of B₀ values corresponding to a plurality of pixels included in the ROI for generating the Z spectrum, or a median value of the plurality of B₀ values, as the resonant frequency of water (0 ppm). Then, the determination function 138 determines the frequency band using the determined resonant frequency of water.

In addition, the determination function 138 may determine a frequency (hereinafter, referred to as a reference frequency) outside the determined frequency band and is not involved (unrelated, has little relevance) in the decrease in the magnetic resonance signal due to the chemical shift, on the basis of information acquired by the acquisition function 146. The MR signal (hereinafter, referred to as a reference MR signal) collected by applying the reference frequency as the saturation pulse is used to normalize a signal value of the contrast Z spectrum and a signal value of the non-contrast Z spectrum.

Specifically, the determination function 138 determines, as the reference frequency, a frequency that is not affected by disturbances such as the saturation frequency of water, a plurality of chemical shifts related to a plurality of CEST substances, the MT effect, the Overhauser effect, and the like. For example, the determination function 138 determines a frequency corresponding to a ppm having an absolute value greater than ±10 ppm (for example, a ppm farther away than ±10 ppm) and corresponding to −20 ppm as the reference frequency. The determination function 138 may determine, as the reference frequency, a frequency corresponding to a position at which the above n is greater than 10. When a saturation pulse is not applied in acquiring the reference MR signal, the determination of the reference frequency by the determination function 138 does not need.

When the reference frequency is determined by the determination function 138, before the subject P is contrasted, the sequence control circuitry 120 uses the determined reference frequency as a saturation pulse to collect a reference MR signal that serves as a reference for the first Z spectrum and the second Z spectrum by the collection function 121. When the reference frequency is not determined by the determination function 138, before the subject P is contrasted, the sequence control circuitry 120 collects the reference MR signal by the collection function 121 without using a saturation pulse. The collection of the reference MR signal may be performed by the contrast CEST imaging, or may be performed by imaging separate from the contrast CEST imaging. The processing circuitry 150 that realizes the determination function 138 corresponds to a decision unit.

The processing circuitry 150, using the calculation function 140, calculates a difference between the first Z spectrum generated on the basis of the first group of magnetic resonance signals and the second Z spectrum generated on the basis of the second group of magnetic resonance signals. For example, the calculation function 140 calculates the difference (hereinafter, referred to as a difference spectrum) by subtracting the second Z spectrum from the first Z spectrum. The calculation function 140 calculates a state quantity in the imaging region (for example, ROI) related to the first Z spectrum and the second Z spectrum on the basis of the plurality of peaks detected by the detection function 142 and the reference MR signal. The state quantity is, for example, temperature or pH in the imaging region. The calculation of pH and the like conforms to a calculation procedure (for example, a ratiometric method) described in, for example, Non-Patent Document, and therefore the description thereof will be omitted. The calculation function 140 stores the calculated state quantity in the storage circuitry 132. The calculated state quantity may be displayed on the display 143, for example, by the control function 133. The processing circuitry 150 that realizes the calculation function 140 corresponds to a calculation unit.

The processing circuitry 150 detects, by the detection function 142, a plurality of peaks indicating a decrease in the magnetic resonance signal due to a chemical shift for a plurality of substances on the basis of the calculated difference. In other words, the detection function 142 detects, on the basis of the difference, a plurality of peaks that correspond to the chemical shifts of each of the plurality of CEST substances and indicate a decrease in the magnetic resonance signal. Specifically, the detection function 142 detects the plurality of peaks by function fitting to the distribution of signal values in the calculated differences or estimation of a contour of the distribution. More specifically, the detection function 412 detects the peaks of the two CEST substances by function fitting to the distribution of the signal values using two approximation equations corresponding to the peaks of the two CEST substances. The detection function 142 may detect the plurality of peaks by the estimation of the contour of the distribution of the signal values (estimation of values of apexes of the peaks) instead of by function fitting. Since known methods can be appropriately used for the function fitting and the estimation of the contour, a description thereof will be omitted. The processing circuitry 150 that realizes the detection function 142 corresponds to a detection unit.

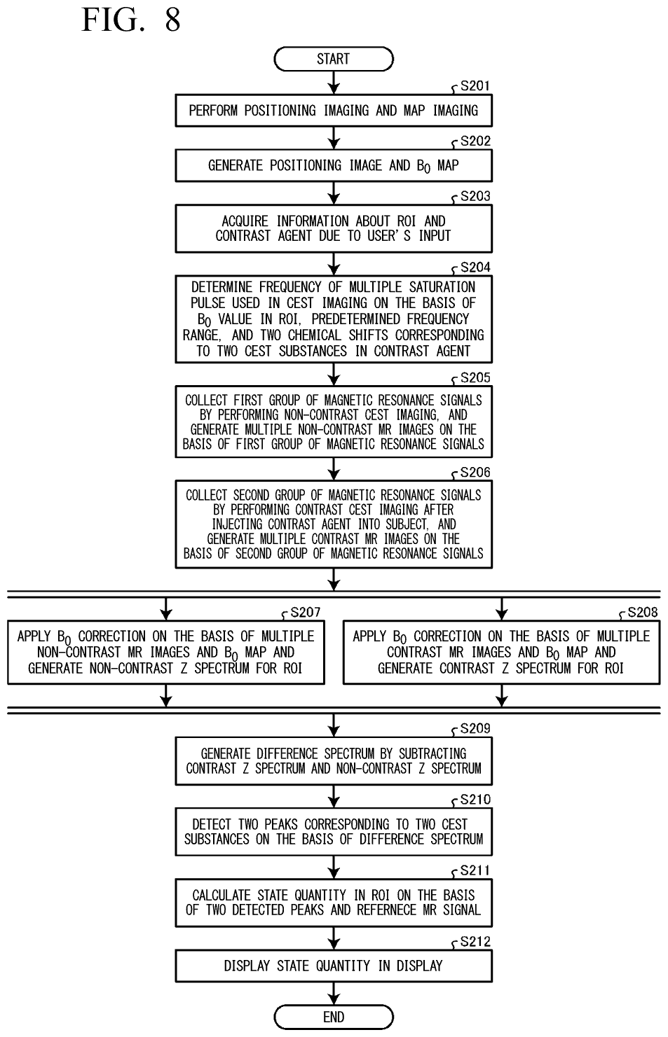

The overall configuration of the MRI apparatus 100 according to the embodiment has been described above. With this configuration, the MRI apparatus 100 according to the embodiment performs non-contrast CEST imaging, map imaging, and contrast CEST imaging, calculates a plurality of chemical shift peaks corresponding to a plurality of substances using MR data collected by each imaging, and performs a process of determining a state quantity on the basis of the plurality of peaks and a reference MR signal (hereinafter, referred to as a state quantity determination process). Hereinafter, a procedure related to the state quantity determination process will be described with reference to FIG. 8. FIG. 8 is a flowchart showing an example of the procedure of the state quantity determination process.

For the sake of concrete explanation, it is assumed below that the static magnetic field strength is 3 T, the predetermined frequency range is 1 ppm, and the contrast agent is iopamidol. In this case, the two CEST substances correspond to two types of substances including two amide groups corresponding to a chemical shift of 4.2 ppm and one amide group corresponding to a chemical shift of 5.6 ppm. A frequency of the saturation pulse of water corresponding to 0 ppm, that is, a center frequency, is 128 MHz. It is also assumed that sequence information regarding the map imaging is set in advance. In addition, sequence information about the non-contrast CEST imaging and the contrast CEST imaging is set in advance, except for the frequencies of the plurality of saturation pulses. Since a known method can be applied to set the sequence information, a description thereof will be omitted. In the state quantity determination process, the same target is imaged in the non-contrast CEST imaging, the map imaging, and the contrast CEST imaging. That is, the imaging target sites in the non-contrast CEST imaging, the map imaging, and the contrast CEST imaging are the same.

The stimulation-imparting mechanism 160 is controlled by the computer 130. The stimulation-imparting mechanism 160 stimulates the contrast agent nanoparticles that have been administered to the subject P and accumulated in a target tissue due to the EPT effect, thereby causing the contrast agent nanoparticles to break down. Since the contrast agent flows out of the broken contrast agent nanoparticles, it becomes possible to acquire a contrast MR image. The stimulation-imparting mechanism 160 corresponds to a stimulation-imparting unit.

### <State Quantity Determination Process>

### (Step S201)

The sequence control circuitry 120 performs positioning imaging and map imaging on the subject P in response to a user instruction via the input device 141. The positioning imaging includes locator imaging and high-speed imaging for setting an ROI. Also, the map imaging may include BI shim imaging or the like. In this step, other pre-imaging may be performed. The collection function 121 collects MR data (hereinafter, referred to as positioning MR data) by the positioning imaging. Also, the collection function 121 collects map MR data by the map imaging.

### (Step S202)

The image generation function 134 generates a positioning image on the basis of the positioning MR data. The control function 133 displays the positioning image, which is also called a locator image, on the display 143. The image generation function 134 generates a B₀ map on the basis of the map MR data. The control function 133 stores the B₀ map in the storage circuitry 132.

### (Step S203)

In response to a user instruction via the input device 141, the acquisition function 136 acquires the ROI in the locator image and information about the contrast agent. This step obtains a position of the ROI for determining the state quantity and a plurality of chemical shifts corresponding to a plurality of CEST substances in the contrast agent. The acquisition function 136 associates the position of the ROI with the plurality of chemical shifts and stores them in the storage circuitry 132. Specifically, the acquisition function 136 acquires two chemical shifts (4.2 ppm and 5.6 ppm) in iopamidol.

### (Step S204)

The determination function 138 determines the frequency band (the frequency of the plurality of saturation pulses), which is used for the CEST imaging, on the basis of a B₀ value in the ROI, a predetermined frequency range, and two chemical shifts corresponding to the two CEST substances in the contrast agent. Specifically, the determination function 138 determines a B₀ value corresponding to a position of the ROI on the basis of the stored position of the ROI and the B₀ map. The determination function 138 reads out the predetermined frequency range from the storage circuitry 132. The determination function 138 determines a frequency band for the plurality of saturation pulses used in the CEST imaging on the basis of the read-out predetermined frequency range and the two chemical shifts corresponding to the two CEST substances. The determination function 138 determines the frequencies of the plurality of saturation pulses on the basis of the determined B₀ value and the frequency band. The determination function 138 determines a reference frequency outside the frequency band on the basis of the two chemical shifts corresponding to the two CEST substances in the contrast agent. The determination function 138 causes the determined frequencies of the saturation pulses, which include the reference frequency, to be stored in the storage circuitry 132.

### (Step S205)

The sequence control circuitry 120 performs the non-contrast CEST imaging on the subject P in response to a user instruction via the input device 141. The collection function 121 collects the first group of magnetic resonance signals by performing the non-contrast CEST imaging. The collection function 121 also collects a reference MR signal corresponding to the reference frequency. The collection function 121 stores the first group of magnetic resonance signals and the reference MR signal in the storage circuitry 132. The image generation function 134 generates a plurality of non-contrast MR images on the basis of the first group of magnetic resonance signals. The image generation function 134 stores the plurality of non-contrast MR images in the storage circuitry 132.

### (Step S206)

After the contrast agent nanoparticles are injected into the subject P, the sequence control circuitry 120 performs the contrast CEST imaging on the subject P in response to a user instruction via the input device 141. The collection function 121 collects the second group of magnetic resonance signals by performing the contrast CEST imaging. At this time, the collection function 121 also collects a reference MR signal corresponding to the reference frequency. The collection function 121 stores the second group of magnetic resonance signals and the reference MR signal in the storage circuitry 132. The image generation function 134 generates a plurality of contrast MR images on the basis of the second group of magnetic resonance signals. The image generation function 134 stores the plurality of contrast MR images in the storage circuitry 132.

In Step S206, as shown in FIG. 1, (A) the contrast agent nanoparticles (also simply referred to as “nanoparticles”) are administered (injected), (B) accumulation of the nanoparticles in a target tissue is monitored, (C) a dissolving stimulus is applied to the nanoparticles to cause them to break down, and (D) the CEST imaging is performed.

The contrast agent nanoparticles injected into the subject P accumulate in the target tissue due to the EPR effect. The accumulation of the contrast agent nanoparticles in the target tissue is monitored, and when sufficient accumulation is reached for imaging, a stimulus is applied to the contrast agent nanoparticles from the stimulation-imparting mechanism 160, causing the contrast agent nanoparticles to break down and to release the incorporated contrast agent.

Monitoring the accumulation of the imaging nanoparticles in a target organ can be accomplished by, but is not limited to, magnetic resonance, x-ray, or visible light.

The stimulus applied to the contrast agent nanoparticles from the stimulus imparting mechanism is not particularly limited as long as it can break the contrast agent nanoparticles, and an electromagnetic field, an electric field, a magnetic field, heat, ultrasound, or the like can be used.

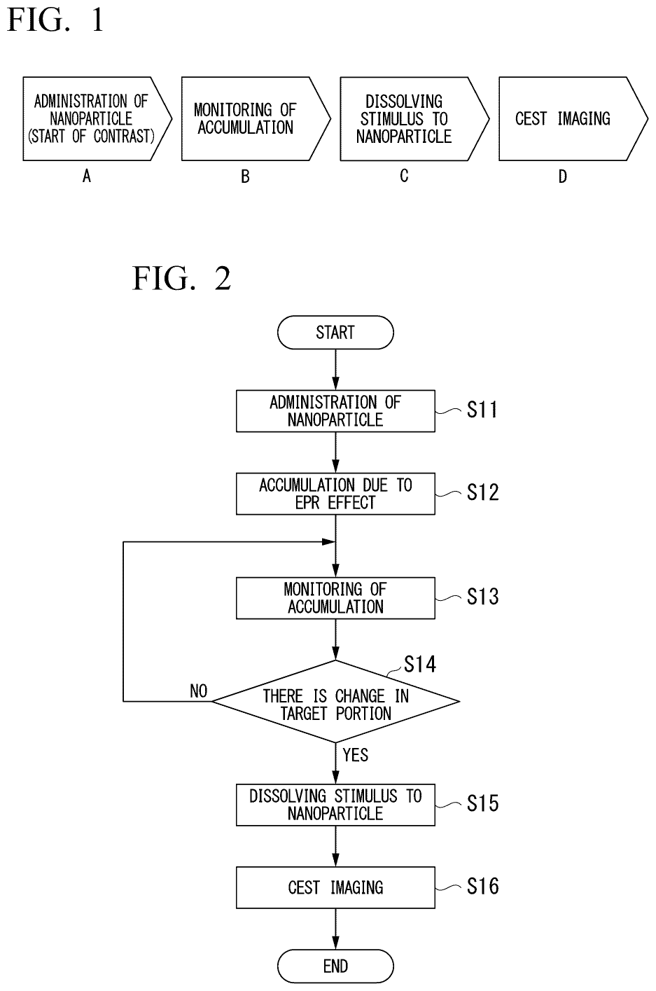

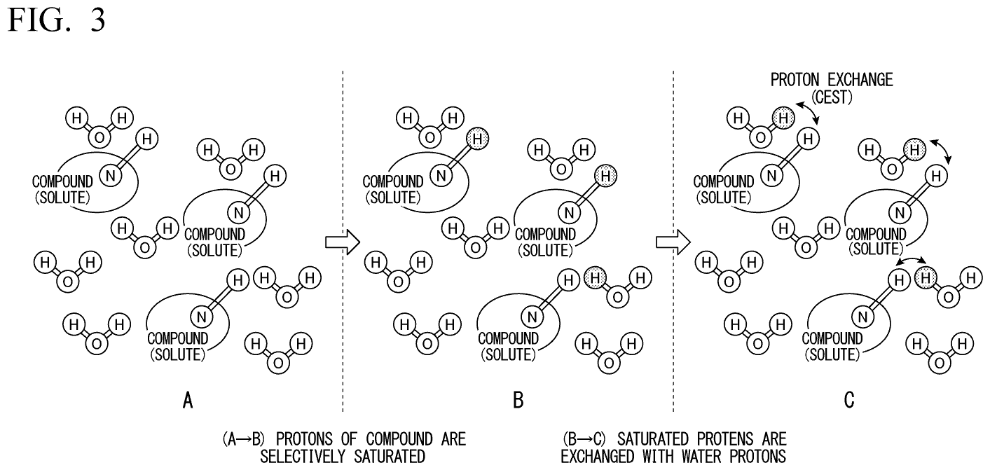

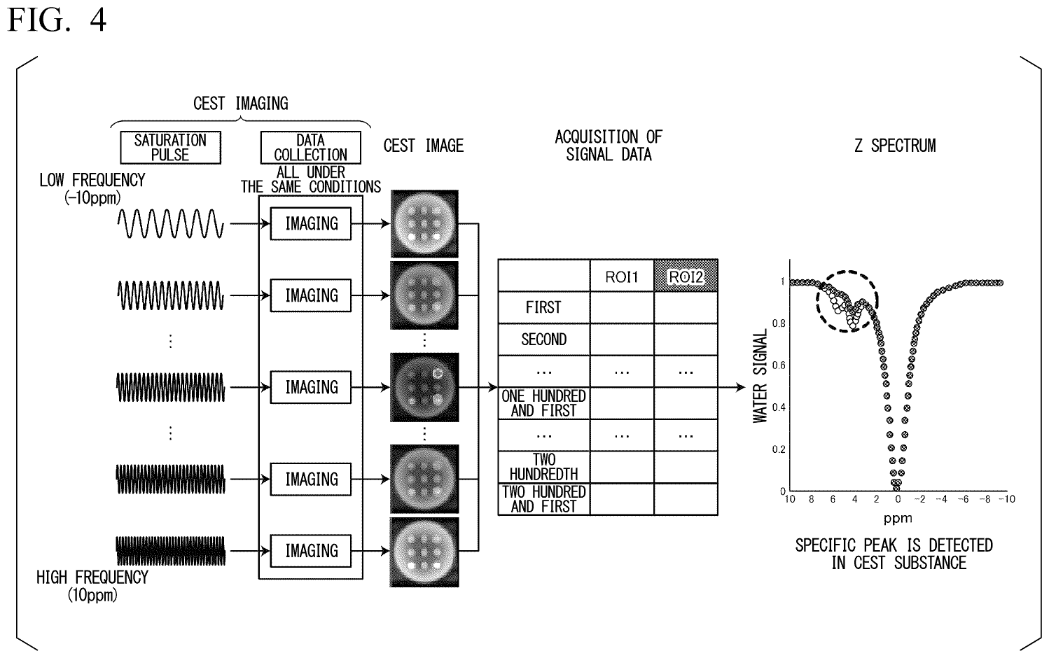

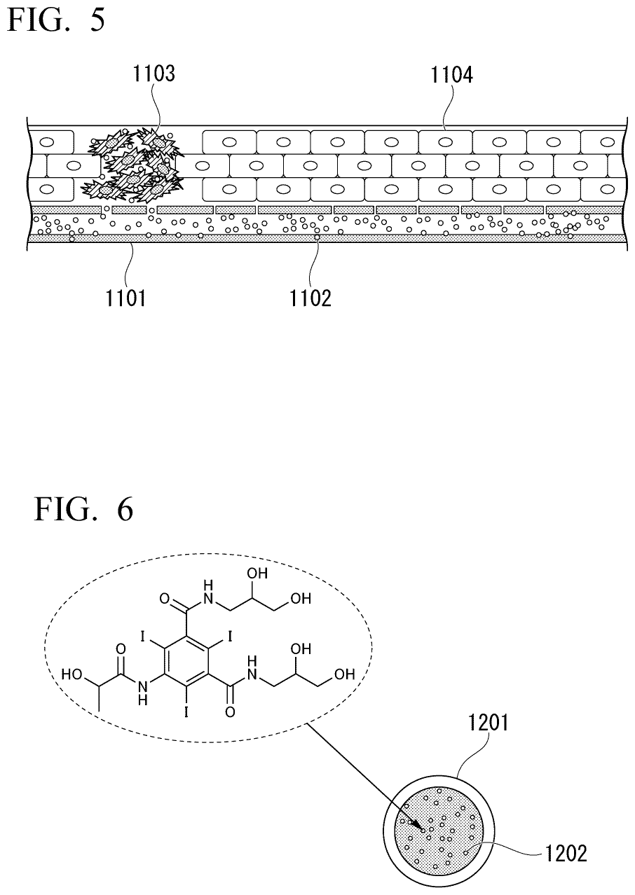

FIG. 2 is a flow chart showing an example of a process from administration (injection) of the nanoparticle contrast agent into the subject P to the CEST imaging.

Step S11: The contrast agent nanoparticles are administered (injected) to the subject P.

Step S12: The contrast agent nanoparticles accumulate in the target tissue due to the EPR effect.

Step S13: The accumulation of the contrast agent nanoparticles in the target tissue is monitored.

Step S14: When there is a change in the target tissue (a target site), Step S15 is performed, and when there is no change, Step S13 is returned to continue monitoring the accumulation of the contrast agent nanoparticles.

Step S15: A dissolving stimulus is applied to the accumulated contrast agent nanoparticles, causing them to dissolve and release the incorporated contrast agent.

Step S16: The CEST imaging is performed.

In one embodiment, the accumulation of the contrast agent nanoparticles in the target tissue is monitored using MRI. Outer surfaces of the contrast agent nanoparticles are preferably modified with metal ions that significantly change a relaxation time to facilitate the monitoring. Such metal ions are preferably alkali metal ions such as lithium ions, sodium ions, or potassium ions. As a monitoring method, it is preferable to continuously capture the change in relaxation time by high-speed imaging. In order to dissolve the contrast agent nanoparticles and to release the incorporated contrast agent, a method suited to a design of the nanoparticles is used, such as irradiating the contrast agent nanoparticles accumulated in the target tissue with an RF pulse in an MRI or irradiating them with heat. In one aspect, by obtaining a concentration of the contrast agent (the CEST substance) incorporated in the contrast agent nanoparticles in advance, it is possible to estimate a concentration of the contrast agent (the CEST substance) released from the contrast agent nanoparticles in the target tissue from an accumulation amount of the contrast agent nanoparticles and a dissolution efficiency due to a dissolving stimulus.

In another aspect, the accumulation of the contrast agent nanoparticles in the target tissue is monitored using an ultrasound imaging device. The outer surfaces of the contrast agent nanoparticles are preferably surface-modified to have different reflectances of (ultra) sound waves to facilitate the monitoring. Such a surface modification includes a modification with metal nanoparticles, polymer nanoparticles, or the like. A preferred monitoring method is to capture a change in reflectance of the (ultra) sonic waves. In order to dissolve the contrast agent nanoparticles and to release the incorporated contrast agent, a method suited to a design of the nanoparticles is used, such as irradiating the contrast agent nanoparticles accumulated in the target tissue with ultrasound, electromagnetic waves, ultraviolet (UV) rays, or infrared (IR) rays. In one aspect, by obtaining the concentration of the contrast agent (the CEST substance) incorporated in the contrast agent nanoparticles in advance, it is possible to estimate the concentration of the contrast agent (the CEST substance) released from the contrast agent nanoparticles in the target tissue from the accumulation amount of the contrast agent nanoparticles and the dissolution efficiency due to a dissolving stimulus.

In another aspect, the accumulation of the contrast agent nanoparticles in the target tissue is monitored using a radiation imaging device. The outer surfaces of the contrast agent nanoparticles are preferably modified to have different radiation transmittance in order to facilitate the monitoring. Such a surface modification includes a modification with metal nanoparticles. A preferred monitoring method is to capture a change in reflectance of (ultra) sonic waves. In order to dissolve the contrast agent nanoparticles and to release the incorporated contrast agent, a method suited to a design of the nanoparticles is used, such as irradiating the contrast agent nanoparticles accumulated in the target tissue with ultrasound, electromagnetic waves, ultraviolet (UV) rays, or infrared (IR) rays. In one aspect, by obtaining the concentration of the contrast agent (the CEST substance) incorporated in the contrast agent nanoparticles in advance, it is possible to estimate the concentration of the contrast agent (the CEST substance) released from the contrast agent nanoparticles in the target tissue from the accumulation amount of the contrast agent nanoparticles and the dissolution efficiency due to a dissolving stimulus.

During a period until the contrast agent nanoparticles accumulate in the target tissue (for example, about 15 to 60 minutes), the sequence control circuitry 120 may perform various types of imaging. For example, during a period immediately after the injection of the contrast agent nanoparticles into the subject P and before the return of contrast CEST imaging, the sequence control circuitry 120 may perform any imaging such as positioning imaging according to an examination order, re-imaging of map imaging, imaging to obtain a T1-weighted image, imaging to obtain a T2-weighted image, EPI, or the like.

### (Step S207)

The image generation function 134 applies a B₀ correction on the basis of the plurality of non-contrast MR images and the B₀ map to generate a non-contrast Z spectrum (the first Z spectrum) for the ROI. The image generation function 134 stores the generated non-contrast Z spectrum in the storage circuitry 132.

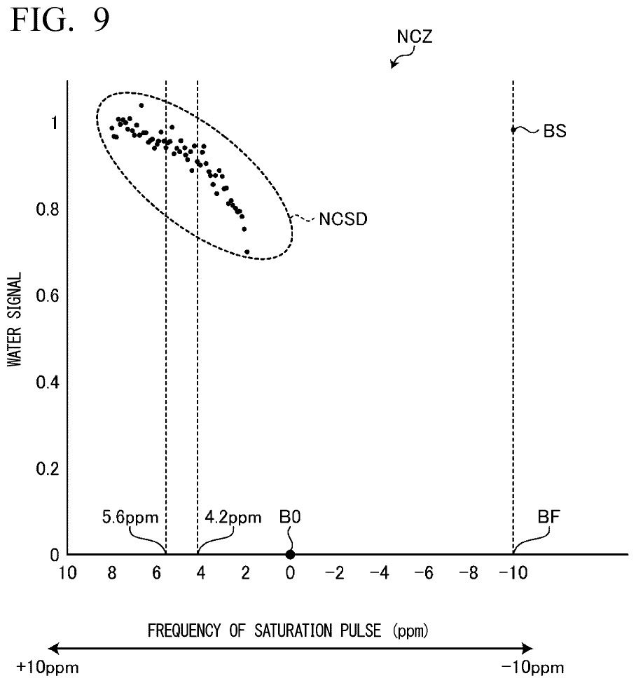

FIG. 9 is a diagram showing an example of a non-contrast Z spectrum NCZ. A distribution of signal values included in a dotted frame NCSD shown in FIG. 9 indicates a distribution of signals of water with respect to the frequency (ppm) of a saturation pulse in the non-contrast state. Also, in the non-contrast Z spectrum NCZ, a reference MR signal BS for a reference frequency BF less than −10 ppm is shown.

### (Step S208)

The image generation function 134 applies the B₀ correction on the basis of the plurality of contrast MR images and the B₀ map to generate a contrast Z spectrum (the second Z spectrum) for the ROI. The image generation function 134 stores the generated contrast Z spectrum in the storage circuitry 132.

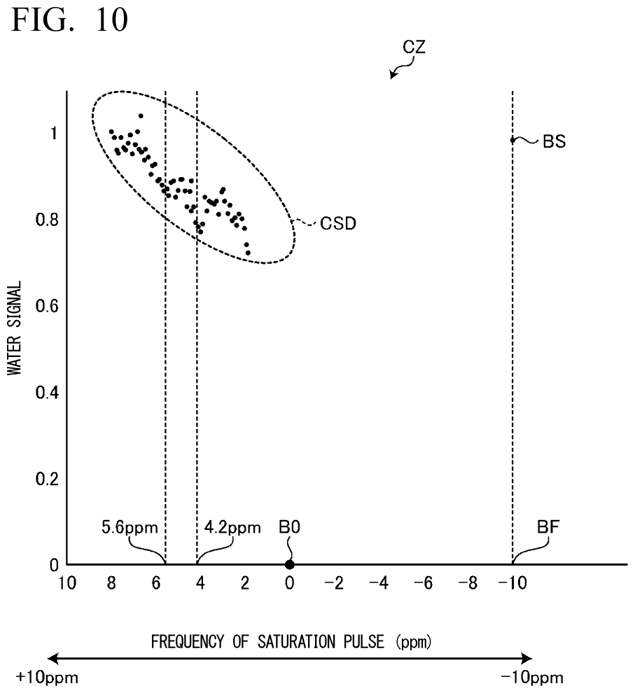

FIG. 10 is a diagram showing an example of a contrast Z spectrum CZ. A distribution of signal values included in a dotted frame CSD shown in FIG. 10 indicates a distribution of signals of water with respect to the frequency (ppm) of a saturation pulse in the contrast Z spectrum CZ. Also, in the contrast Z spectrum CZ, a reference MR signal BS for a reference frequency BF smaller than −10 ppm is shown. Also, 0 ppm B₀ in the contrast Z spectrum CZ corresponds to a frequency determined by the B₀ correction. When the collection of the reference MR signal BS is performed on either the non-contrast CEST imaging or the contrast CEST imaging, the reference MR signal BS is, for example, not displayed in the non-contrast Z spectrum NCZ and the contrast Z spectrum CZ.

### (Step S209)

The calculation function 140 subtracts the contrast Z spectrum CZ from the non-contrast Z spectrum NCZ to generate a difference spectrum. Specifically, the calculation function 140 generates the difference spectrum by subtracting the contrast Z spectrum CZ from the non-contrast Z spectrum NCZ. The calculation function 140 stores the generated difference Z spectrum in the storage circuitry 132.

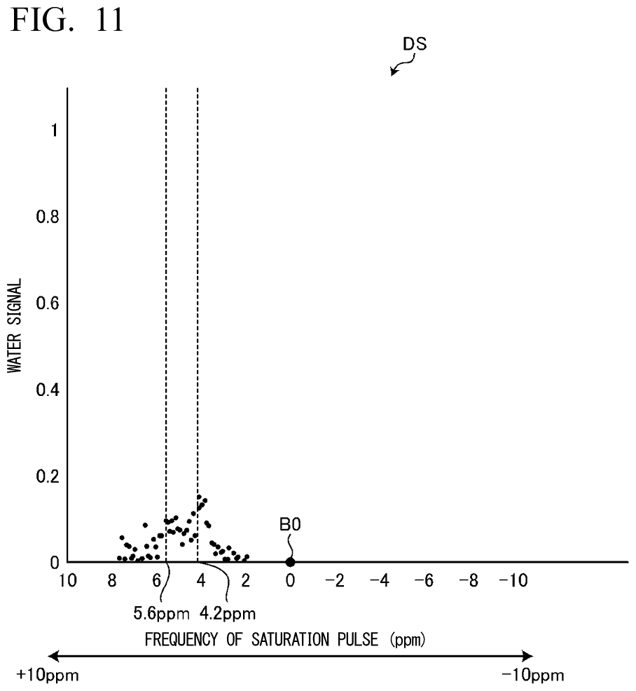

FIG. 11 is a diagram showing an example of a difference spectrum DS. As shown in FIG. 11, the difference spectrum DS includes a distribution of signal values of signals of water having peaks at two chemical shifts (4.2 ppm and 5.6 ppm) in iopamidol.

### (Step S210)

The detection function 142 detects two peaks corresponding to the two CEST substances on the basis of the difference spectrum DS. For example, the detection function 142 performs function fitting or an estimation of the contour of the distribution of signal values in the difference spectrum DS. Thus, the detection function 142 detects two peaks corresponding to the two CEST substances in the difference spectrum DS. The detection function 142 stores the two detected peaks in the storage circuitry 132.

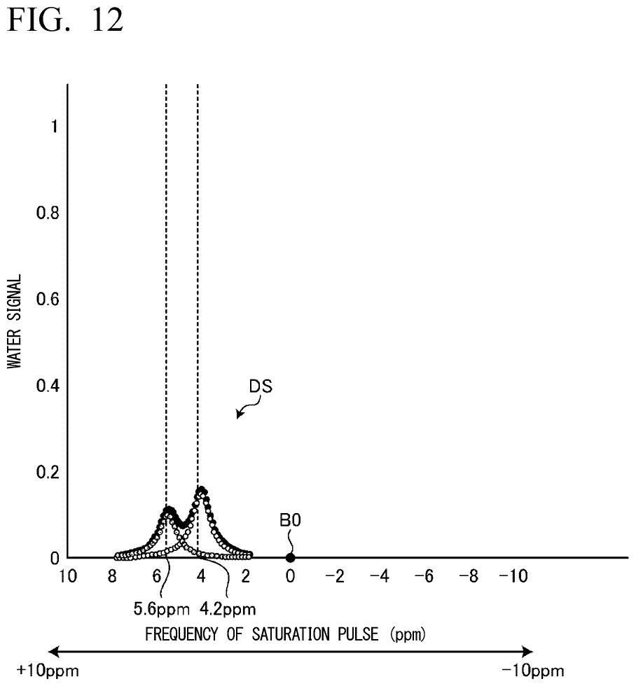

FIG. 12 is a diagram showing an example in which function fitting is performed on the difference spectrum DS. As shown in FIG. 12, the detection function 142 detects peaks at 4.2 ppm and 5.6 ppm by applying a fitting process to the difference spectrum DS using a function indicating 4.2 ppm and a function indicating 5.6 ppm.

The detection of the peak is not limited to the above. For example, when a plurality of difference spectra are generated, the detection function 142 integrates a plurality of signal values at five points (two peak ppms, intermediate ppm, and two interval ppms) including ppm related to a plurality of peaks (hereinafter, referred to as peak ppms), ppm located midway between adjacent peak ppms (hereinafter, referred to as intermediate ppm), and ppm at two positions separated from each of the two peak ppms by a distance between the adjacent peak ppm in a direction opposite to a direction from each of the two peak ppms toward the intermediate ppm (hereinafter, referred to as separated ppm). That is, the detection function 142 averages a plurality of signal values at each of the five points in the plurality of difference spectra.

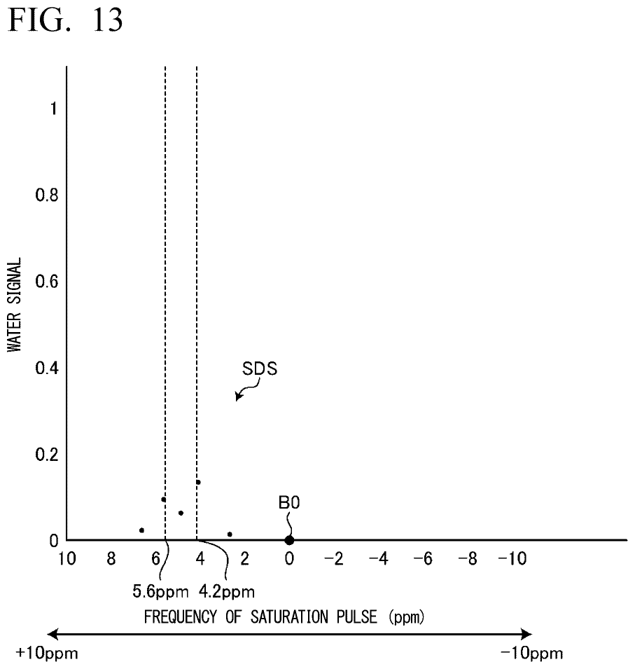

FIG. 13 is a diagram showing an example of a difference spectrum SDS after integration. When a contrast agent contains two CEST substances having two peaks, the difference spectrum after integration has five points as shown in FIG. 13. The detection function 142 estimates signal values of two peaks for the difference spectrum SDS after integration shown in FIG. 13. For example, the detection function 142 detects peaks at 4.2 ppm and 5.6 ppm by performing function fitting or an estimation of a contour of the distribution on the points. In the estimation of the contour, for example, the two peaks are detected by applying the signal values at two peak ppms (4.2 ppm and 5.6 ppm) as maximum values, the intermediate ppm as a minimum value, and the two separated ppms as initial values in the vicinity of a point of contact on the horizontal axis to two approximation equations corresponding to the peaks of the two CEST substances

### (Step S211)

The calculation function 140 calculates the state quantity in the ROI on the basis of the two detected peaks and the reference MR signal. The calculation function 140 stores the calculated state quantity in the storage circuitry 132.

### (Step S212)

The control function 133 causes the display 143 to display the calculated state quantity.

The MRI apparatus 100 according to the embodiment described above acquires information about a contrast agent including a plurality of substances having different chemical shifts, determines a frequency band related to a decrease in a magnetic resonance signal due to the chemical shift on the basis of the acquired information, collects a first group of magnetic resonance signals by CEST (chemical exchange saturation transfer) imaging while changing conditions of a saturation pulse for the determined frequency band before the contrast agent is applied, collects a second group of magnetic resonance signals by CEST imaging while changing conditions of the saturation pulse for the frequency band after the contrast agent is applied, calculates a difference between a first Z spectrum generated on the basis of the first group of magnetic resonance signals and a second Z spectrum generated on the basis of the second group of magnetic resonance signals, and detects a plurality of peaks which correspond to the chemical shifts of each of the plurality of substances and indicate the decrease in the magnetic resonance signal on the basis of the calculated difference.

In the MRI apparatus 100 according to this embodiment, the first group of magnetic resonance signals and the second group of magnetic resonance signals correspond to a plurality of MR signals collected in a localized frequency band compared to the frequency band of a conventional saturation pulse. Moreover, in the MRI apparatus 100 according to this embodiment, the first Z spectrum and the second Z spectrum correspond to local Z spectra compared to a conventional Z spectrum (full spectrum).

Moreover, the MRI apparatus 100 according to the embodiment further uses a B₀ map generated before the execution of the CEST imaging to determine the frequency band. Moreover, the MRI apparatus 100 according to the embodiment detects a plurality of peaks by performing function fitting on the distribution of signal values in the calculated difference or estimating a contour of the distribution. In addition, the MRI apparatus 100 according to the embodiment determines a frequency outside the frequency band to be determined and is not involved in the decrease in the magnetic resonance signal due to the chemical shift, on the basis of the acquired information, and collects a reference MR signal as a reference for the first Z spectrum and the second Z spectrum, using the determined frequency as a saturation pulse or without using a saturation pulse, and the first Z spectrum is generated on the basis of the first group of magnetic resonance signals and the reference MR signal, and a second Z spectrum is generated on the basis of the second group of magnetic resonance signals and the reference MR signal.

Moreover, the MRI apparatus 100 according to the embodiment calculates state quantities in imaging regions related to the first Z spectrum and the second Z spectrum on the basis of the detected plurality of peaks and the reference MR signal. In the MRI apparatus 100 according to the embodiment, the calculated state quantity is temperature or pH in the imaging region.

For these reasons, according to the MRI apparatus 100 according to the embodiment, it is possible to realize a fitting method that can reduce the influence of unconsidered parameters in a living body (such as initial values of a fitting function for each site), and can also reduce imaging time and initial conditions with respect to detection of peaks of decline in signal values for a plurality of CEST substances. Thus, according to the MRI apparatus 100 according to the embodiment, by significantly reducing the time required for calculations relating to the signal values of the peaks and the amount of data required for the Z spectrum used in the calculations, the time required for imaging can be reduced while the accuracy of the data related to the peaks can be improved.

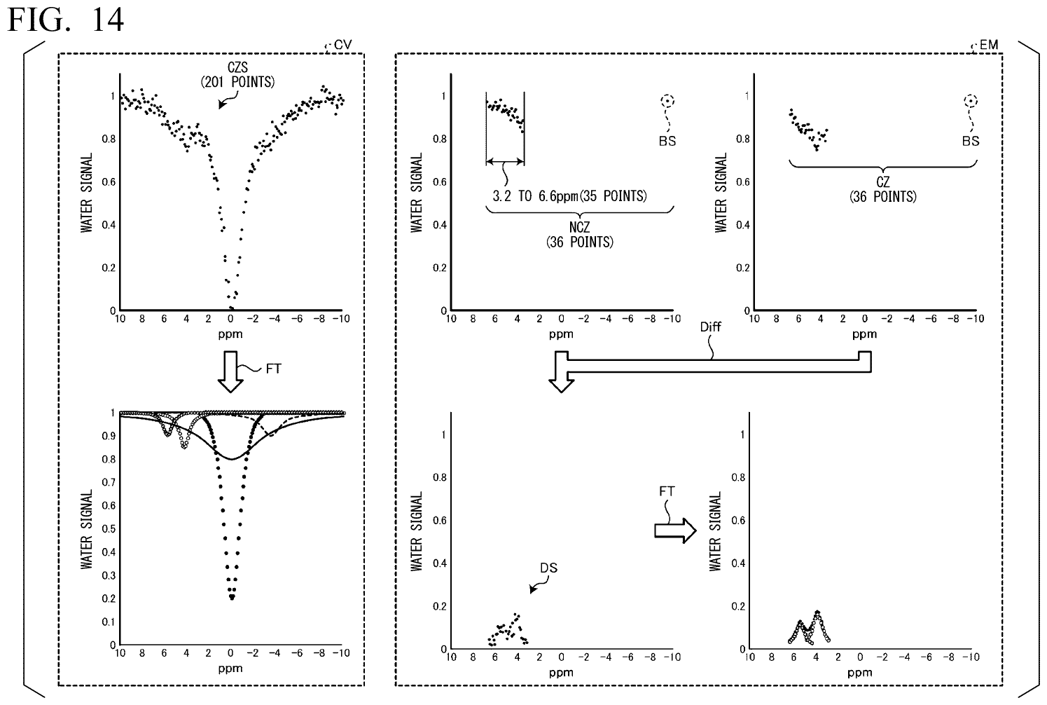

FIG. 14 is a diagram showing an example of the effect of this embodiment. In a conventional CV technique for acquiring a full spectrum in increments of 0.1 ppm in a frequency range of the saturation pulse from −10 ppm to 10 ppm, 201 images are acquired. At this time, fitting FT is performed on a Z spectrum CZS in the conventional CV using five approximate equations. On the other hand, in the EM of this embodiment, it is possible to collect (2n+1+1) images centered on the chemical shifts of each of the two CEST substances, and for example, when n=10, a total of 88 images will be collected before and after a contrast agent is applied. Therefore, according to the MRI apparatus 100 of this embodiment, imaging can be performed in approximately half the time compared to the conventional method.

As shown in FIG. 14, when iopamidol was used as a contrast agent, the number of CEST imaging operations, including the reference frequency and centered on the chemical shifts (4.2 ppm and 5.6 ppm) of the two CEST substances, is 72 points (36 points+36 points) in total, including before and after the contrast agent. That is, according to the MRI apparatus 100 of this embodiment, the number of imaging operations is reduced to half or less compared to the conventional apparatus. Further, as shown in FIG. 14, in the fitting FT to a difference spectrum DS generated by a difference Diff between the non-contrast Z spectrum NCZ and the contrast Z spectrum CZ, the number of boundary conditions and fitting curves is reduced from five to two compared to the conventional method, thereby improving the accuracy of the fitting.

From the above, according to the MRI apparatus 100 of the embodiment, since it is possible to reduce the imaging time and the initial conditions (the boundary conditions) required for fitting, and it is possible to obtain a highly accurate peak signal value in a shorter time than in the conventional apparatus, the state quantity in the ROI can be calculated with high accuracy and in a short time.

When the technical idea of the embodiment is realized in a chemical shift peak detection method, the chemical shift peak detection method acquires information about a contrast agent including a plurality of substances (CEST substances) having different chemical shifts, determines a frequency band related to a decrease in a magnetic resonance signal due to the chemical shift on the basis of the acquired information, collects a first group of magnetic resonance signals by CEST (chemical exchange saturation transfer) imaging while changing conditions of a saturation pulse with respect to a frequency band before a contrast agent is applied, collects a second group of magnetic resonance signals by CEST imaging while changing the conditions of the saturation pulse with respect to a frequency band after the contrast agent is applied, calculates a difference between a first Z spectrum generated on the basis of a first group of magnetic resonance signals and a second Z spectrum generated on the basis of a second group of magnetic resonance signals, and detects a plurality of peaks corresponding to the plurality of substances and indicating the decrease in the magnetic resonance signal on the basis of the calculated difference.

The contrast agent containing the CEST substance is administered to the subject to be imaged in the form of nanoparticles containing the contrast agent (contrast agent nanoparticles) before the contrast agent is applied. The contrast agent nanoparticles administered to the subject to be imaged accumulate in an imaging target site, such as a tumor, within the subject's body due to the enhanced permeability and retention (EPR) effect. The accumulation of the contrast agent nanoparticles in the imaging target site is monitored, and when an amount of accumulation of the CEST substance becomes sufficient for CEST imaging, a stimulus is applied to the contrast agent nanoparticles to dissolve the nanoparticles. Then, the CEST imaging is performed.

The chemical peak shift detection method according to the embodiment applies a dissolving stimulus to the contrast agent nanoparticles accumulated in an imaging region of the subject, collects a first group of magnetic resonance signals by CEST imaging while changing conditions of a saturation pulse before being contrasted with the contrast agent nanoparticles, collects a second group of magnetic resonance signals by the CEST imaging while changing the conditions of the saturation pulse after being contrasted with the contrast agent nanoparticles and after the dissolving stimulus is applied to the contrast agent nanoparticles, calculates a difference between a first Z spectrum generated on the basis of the first group of magnetic resonance signals and a second Z spectrum generated on the basis of the second group of magnetic resonance signals, and detects a plurality of peaks indicating a decrease in the magnetic resonance signal due to a chemical shift on the basis of the difference.

Since the procedure and effects of the state quantity determination process, including the procedure of the chemical shift peak detection method, are similar to those in the embodiment, the description thereof will be omitted.

According to at least one of the embodiments described above, it is possible to reduce the imaging time and to improve the detection accuracy of the chemical shift peak.

Although several embodiments have been described, these embodiments are presented as examples and are not intended to limit the scope of the invention. These embodiments can be implemented in various other forms, and various omissions, substitutions, modifications, and combinations of the embodiments can be made without departing from the spirit of the invention. These embodiments and variations thereof are included within the scope of the invention and equivalents thereof as described in the claims, as well as within the scope and spirit of the invention.

What is claimed is:

1. A magnetic resonance imaging apparatus comprising:
a stimulation-imparting unit;
sequence control circuitry; and
processing circuitry,
wherein the stimulation-imparting unit is configured to apply a dissolving stimulus to contrast agent nanoparticles accumulated in an imaging region after the contrast agent nanoparticles are injected into a subject;
wherein the sequence control circuitry is configured to:
collect a first group of magnetic resonance signals by CEST imaging while changing conditions of a saturation pulse before being contrasted with the contrast agent nanoparticles; and
collect a second group of magnetic resonance signals by the CEST imaging while changing the conditions of the saturation pulse after being contrasted with the contrast agent nanoparticles and after the dissolving stimulus by the stimulation-imparting unit is applied,
wherein the processing circuitry is configured to:
calculate a difference between a first Z spectrum generated on the basis of the first group of magnetic resonance signals and a second Z spectrum generated on the basis of the second group of magnetic resonance signals; and
detect a plurality of peaks indicating a decrease in a magnetic resonance signal due to a chemical shift on the basis of the difference.

2. The magnetic resonance imaging apparatus according to claim 1, wherein the processing circuitry is further configured to:
acquire information about a contrast agent including a plurality of substances having different chemical shifts; and
determine a frequency band related to the decrease in the magnetic resonance signal due to the chemical shift on the basis of the information.

3. The magnetic resonance imaging apparatus according to claim 2, wherein the processing circuitry is configured to determine the frequency band by further using a B₀ map generated before the CEST imaging is performed.

4. The magnetic resonance imaging apparatus according to claim 1, wherein the processing circuitry is configured to detect the plurality of peaks by performing function fitting to a distribution of signal values in the difference or estimating a contour of the distribution.

5. The magnetic resonance imaging apparatus according to claim 2, wherein the processing circuitry is configured to determine, on the basis of the information, a frequency outside the frequency band and is not involved in the decrease in the magnetic resonance signal due to the chemical shift,
wherein the sequence control circuitry is configured to collect a reference MR signal as a reference for the first Z spectrum and the second Z spectrum, using the determined frequency as the saturation pulse or without using the saturation pulse,
the first Z spectrum is generated on the basis of the first group of magnetic resonance signals and the reference MR signal, and
the second Z spectrum is generated on the basis of the second group of magnetic resonance signals and the reference MR signal.

6. The magnetic resonance imaging apparatus according to claim 5, wherein the processing circuitry is configured to calculate a state quantity in an imaging region related to the first Z spectrum and the second Z spectrum on the basis of the plurality of peaks and the reference MR signal.

7. The magnetic resonance imaging apparatus according to claim 6, wherein the state quantity is temperature or pH in the imaging region.

8. A chemical shift peak detection method comprising:
applying a dissolving stimulus to contrast agent nanoparticles accumulated in an imaging region of a subject;
collecting a first group of magnetic resonance signals by CEST imaging while changing conditions of a saturation pulse before being contrasted with the contrast agent nanoparticles;
collecting a second group of magnetic resonance signals by the CEST imaging while changing the conditions of the saturation pulse after being contrasted with the contrast agent nanoparticles and after the dissolving stimulus is applied to the contrast agent nanoparticles;
calculating a difference between a first Z spectrum generated on the basis of the first group of magnetic resonance signals and a second Z spectrum generated on the basis of the second group of magnetic resonance signals; and
detecting a plurality of peaks indicating a decrease in a magnetic resonance signal due to the chemical shift on the basis of the difference.

9. The chemical shift peak detection method according to claim 8, further comprising before the dissolving stimulus is applied to the contrast agent nanoparticles, acquiring information about a contrast agent including a plurality of substances having different chemical shifts, and determining a frequency band related to a decrease in a magnetic resonance signal due to the chemical shift on the basis of the information.

10. The chemical shift peak detection method according to claim 9, further comprising after the first group of magnetic resonance signals is collected, monitoring accumulation of contrast agent nanoparticles in a target tissue, the contrast agent nanoparticles being formed by incorporating a contrast agent containing the plurality of substances having different chemical shifts in stimulus-sensitive nanoparticles; and
when the contrast agent nanoparticles have accumulated in the target tissue to an extent sufficient for the CEST imaging, applying a dissolving stimulus to the contrast agent nanoparticles to release the incorporated contrast agent so that the target tissue is contrasted.

11. The chemical shift peak detection method according to claim 8, wherein the accumulation of the contrast agent nanoparticles in the target tissue is monitored using a magnetic resonance imaging device, a radiation imaging device, or an ultrasound imaging device.

12. The chemical shift peak detection method according to claim 8, wherein the dissolving stimulus to the contrast agent nanoparticles is RF pulse irradiation, electromagnetic wave irradiation, ultrasonic irradiation, radiation irradiation, ultraviolet irradiation, infrared irradiation, or heat irradiation.

13. A contrast agent nanoparticle formed by incorporating a contrast agent containing a plurality of substances having different chemical shifts in a stimulus-sensitive nanoparticle.

14. The contrast agent nanoparticle according to claim 13, wherein the contrast agent is iopamidol.

15. The contrast agent nanoparticle according to claim 13, wherein the stimulus-sensitive nanoparticle is a liposome.

16. The contrast agent nanoparticle according to claim 15, wherein an outer surface of the liposome is modified with a polymer, a metal, a metal complex, or a metal ion.
